(19) United States
(12) Patent Application Publication
(10) Pub. No.: US 2007/0173912 A1
(43) Pub. Date: Jul. 26, 2007

(54) Phototherapy with specific infrared light for treating skin disorders

(76) Inventor: Somnuk Amornsiripanitch, Nonthaburee (TH)

Correspondence Address: FISH & RICHARDSON PC, P.O. BOX 1022, MINNEAPOLIS, MN, 55440-1022, US

(21) Appl. No.: 11/394,672

(22) Filed: Mar. 31, 2006

Related U.S. Application Data
(60) Provisional application No. 60/760,507, filed on Jan. 20, 2006.

(51) Int. Cl. A61N 5/00

(52) U.S. Cl. 607/86

(57) ABSTRACT

Methods of treating skin disorders, specifically the entity named psoriasis, as well as chronic dermatitis and lichen simplex chronicus, involve cooling an exposed surface of a region afflicted with the disorder and applying energy, for example, energy from a coherent or incoherent light source, to the region.

### CROSS-REFERENCE TO RELATED APPLICATIONS

This patent application claims benefit from U.S. Provisional Patent Application No. 60/760,507, filed Jan. 20, 2006, the complete disclosure of which is incorporated herein by reference.

### TECHNICAL FIELD

This disclosure relates generally to methods of treating mammalian skin disorders.

### BACKGROUND

A variety of skin disorders can afflict mammals, in particular, humans. For example, papulosquamous disorders can be identified clinically by the thickening of epidermis and the formation of scales on the top of lesions. In particular, in psoriasis, the thickening of epidermis and the silver scales are accompanied by skin inflammation (redness and abscess). Examples of other mammalian skin disorders include chronic dermatitis and lichen simplex chronicus.

Although some currently available treatments can alleviate mammalian skin disorders, they generally result in some side effects. The need for the development of treatments for skin disorders with minimal side effects is pronounced.

### SUMMARY

The present disclosure addresses problems of skin disorders and of the various treatment options that still have some serious and long-term side effect.

In one aspect, the disclosure features a method of treating skin disorders in a preselected region of human skin, scalp and nails having at least one lesion characteristic of the disorder disposed therein. The method involves cooling an area of the skin above the preselected skin region, and applying energy to the preselected region, in the absence of an exogenously provided energy absorbing material, in an amount sufficient to ameliorate the lesion, while keeping the temperature of the area of the skin above the preselected region below about 60 degrees Celsius before, during, or before and during the application of the energy. In a preferred embodiment, the treatment is performed with the Smoothbeam® laser system. In one embodiment, the treatment can be combined with other methods of treatment of skin disorders.

In one embodiment, application of the energy can be provided by laser light or incoherent light. In a preferred embodiment, the application of the energy can be provided by laser light, e.g., the laser light with a wavelength in the range from about 1.4 microns to about 2.5 microns or, e.g., the laser light with a wavelength in the range from about 1.4 microns to about 1.5 microns. The laser light can further have a fluence in the range from about 2 joules to about 500 joules per square centimeter. The laser light can further have a duration in the range of 1.0 millisecond to 300 milliseconds. In one embodiment, the laser light can also have its duration parted into multiple subpulses, e.g., 4-5 subpulses. The multiple subpulses can have a duration range from e.g., 1 to 60 milliseconds, and the interval between each subpulse can be, e.g., 2-6 milliseconds.

In one embodiment, the disclosure features the method where the cooling of an area of the skin above the preselected region occurs before the application of energy. In another embodiment, the cooling of an area of the skin above the preselected region occurs contemporaneously with the application of energy.

In a preferred embodiment, the skin disorder treated is psoriasis. In a further embodiment, itch of psoriasis can be treated. The treatment of psoriasis, e.g., treatment of psoriasis itch, can be combined with other known psoriasis treatments. In a preferred embodiment, the treatment of psoriasis, e.g., treatment of psoriasis itch, can be performed with the Smoothbeam® laser system.

In one embodiment, the treatment, e.g., Smoothbeam® laser system treatment, can be given once, once a week, two times per week, three times per week, every week, biweekly, tri-weekly or once a month. The treatment can be combined with other skin disorder treatments, e.g., with other treatments for skin disorders, e.g., with other treatments for psoriasis, e.g., with other treatments for psoriasis itch.

In one embodiment, the skin disorder treated is chronic dermatitis. In a preferred embodiment, the treatment of chronic dermatitis can be performed with the use of Smoothbeam® laser system. The treatment with Smoothbeam® can be further combined with other known chronic dermatitis treatments.

In another embodiment, the skin disorder treated is lichen simplex chronicus. In a preferred embodiment, the treatment of lichen simplex chronicus can be performed with Smoothbeam® laser system. The treatment with Smoothbeam® can be further combined with other known lichen simplex chronicus treatments.

The details of one or more embodiments of the disclosure are set forth in the description below. Other features, objects, and advantages of the disclosure will be apparent from the description and from the claims.

### DETAILED DESCRIPTION

Psoriasis

Psoriasis is a papulosquamous skin disorder, which can be identified by the thickening of the epidermis and the formation of scales on top of the lesions, accompanied by inflammation.

The areas affected by psoriasis include scalp, face, body, arms, legs, nails etc. Psoriasis can occur as a few lesions or can be widely distributed over the whole body. Psoriasis can present itself in many forms, such as plaque-type, guttate, inverse and erythrodermic. It often appears between the ages of 15-35, but can develop at any age. In rare cases, it can affect infants. An estimated 2-3% of world's population is affected by psoriasis.

In the United States, 4.5 million adults have been diagnosed with psoriasis. About 30% of the affected individuals have the severe types of psoriasis. Nearly 2.4 million psoriasis patients visit dermatologists each year in United States. Overall costs of treating psoriasis may exceed $3 billion annually. From 150,000 to 260,000 cases of psoriasis are diagnosed each year.

Well-known treatments for psoriasis include topical steroid creams and ointments. The specific topical treatments also include coal tar, anthralin, vitamin D3 (Dovonex) and Protopic ointment. Systemic medications to treat psoriasis include metofrexate, oral retinoid, cyclosporine, mycophenolate mofetil, sulfasalazine and 6-Thioguanine. Another type of systemic treatment for psoriasis includes biologic drugs such as Amevive, Enbrel, Humira, Raptiva and Remicade.

Other approaches to treat psoriasis include the uses of light treatment, generally called phototherapy. Most of the uses of light energy to treat psoriasis are performed in non-ablative mode. Lights in the wavelength of ultraviolet A and ultraviolet B are the main energy for the treatment of psoriasis skin. Few studies mention some benefit of using other wavelengths of light to non-ablatively ameliorate psoriasis with some improvements, such as the use of pulse dye laser (585-595 nm), or infrared laser (1320 nm; reported by Ruiz-Esparza in Dermatol. Surg. 1999 May; 25(5):403-7). The ablative technique use of light was also reported in treatment of psoriasis, for example the use of Er:YAG laser reported by Boehncke W H et al. in Dermatol. Surg. 1999 August; 25(8):618-21. Among the mentioned phototherapies for psoriasis, the excimer laser (UVB 308 nm) is the most effective and has received much attention, since the use of the laser system can limit the exposure of normal skin to the ultraviolet light, which can cause skin cancer.

Smoothbeam® (the product of Candela Corporation, Wayland, Mass.), is the 1450 nm laser system approved by the U.S. Food and Drug Administration to treat acne vulgaris, acne scars and facial wrinkles. It has been observed that the inflammation in the acne lesion can dry up well under the infrared laser treatment, and the acne comedone can push itself out easier. This laser system also can remodel the dermal structure.

Smoothbeam® can also be used to effectively treat folliculitis on the human scalp because its wavelength is not absorbed by melanin pigment. It has also been observed that the 1450 nm wavelength of the Smoothbeam® does not suppress the hair growth, as measured by the method described in U.S. Pat. No. 6,389,150.

The present disclosure provides, in one embodiment, a method of phototherapy to treat psoriasis by using infrared light in the wavelength between 1400 nm to 2500 nm (1.4 microns to 2.5 microns). The light energy can be incoherent or coherent (e.g., a laser). In one embodiment, energy in the infrared range can be absorbed into the psoriatic lesions and hasten the healing process of the lesion. The applied energy can disrupt the normal habitat for the survival of dermal inflammatory cells, destroy the functions of cytokines and terminate inflammatory cells, such as neutrophils and lymphocytes directly. The applied energy can also affect the epidermis by drying the inflammation in the epidermis and enhancing the shedding of affected epidermal layers.

To prevent the epidermis in the lesion from burning with high energy of infrared light, in one embodiment the cooling system can be added prior to, or concomitantly with, the use of the energy. Because the infrared light is safer than the ultraviolet light with regard to causing skin cancer, the application of infrared light can be repeated more often than the application of ultraviolet light. The treatment of the disclosure can be provided, e.g., as often as three times per week with the excimer laser, or it can be provided, e.g., biweekly or, e.g., monthly. In one embodiment, the disclosed treatment can be used in the combination with other topical, systemic treatments and other phototherapy using the well-known light energy. The preferred embodiment utilizes Smoothbeam® to treat psoriasis. In the preferred embodiment, the energy setting is between 2-14 j/cm2 with dynamic cooling device (DCD) set for 20-40 milliseconds. In one embodiment, the energy is applied to the whole area of the lesion through the hand-piece aperture.

Itch in Psoriasis

Itch can be a serious problem in psoriasis. As discussed in Dawn A., “Practical Approaches to Relieving Itch in Psoriasis” at http://www.livingwithitch.org/articles/psoriasis/index.html (the contents of which are incorporated herein by reference), chronic itch leads to chronic scratching and development of new psoriasis plaques. Treatments available for controlling itch in psoriasis include moisturizers, topical salicylates, topical steroids, tar products, agents that induce cool sensation, capsaicin, topical immunomodulators, antidepressants, and biologics (see Dawn A., throughout). Psoriasis, however can become resistant to medications (Dawn, Conclusion).

We found that Smoothbeam® laser system can help stop itching symptoms in psoriasis.

Other Skin Disorders

Dermatitis is a chronic skin disorder characterized by scaly and itching rashes. It is often accompanied by blisters with oozing and crusting.

Lichen simplex chronicus is a skin disorder characterized by chronic itching and scratching. It may be associated with chronic dermatitis (eczema), psoriasis or psychologic disorders. Its symptoms include scaling, lesions and leathery skin (lichenification).

Both dermatitis and lichen simplex chronicus have severe itch symptoms. The itch symptoms can be alleviated by the disclosed methods.

These, and other skin disorders can be treated with the disclosed methods.

### Example 1

### Treating Scalp Psoriasis

The Smoothbeam® laser system was used to treat patients with psoriatic scalp with the dosage of 8-12 J/cm2 and DCD set at 20-40 milliseconds. Some patients underwent systemic treatments but at very low dosages. The results were dramatic; two weeks after the laser treatment, the lesions demonstrated minimal inflammation, no scales and negligible induration (dermal thickening).

### Example 2

### Treating Severe Plaque-Type Psoriasis

The Smoothbeam® laser system was also used to treat the severe plaque-type psoriasis. Ranges of energy and of cooling similar to those used in Example 1 were used on 2 arms and 1 leg of a patient, who was concurrently treated with cyclosporine at 25 mg/day, vitamin B3 and topical 0.1% of triamcinolone lotion. At a one to two week follow-up, the treated limbs demonstrated dramatic improvement. The untreated leg demonstrated some improvement but still, significantly, had some inflammation. After the follow-up, the patient stopped taking cyclosporine, vitamin B3 and triamcinolone lotion and new lesions were treated only with the Smoothbeam® laser system with excellent results.

After the improvement of the body lesions, the patient was also treated on her psoriatic nails. Her psoriatic fingernails demonstrated onycholysis pattern, and her psoriatic toenails demonstrated subungual hyperkeratotic pattern. After the treatment with Smoothbeam® at the dosage of 10-11 J/cm2 and DCD at 20-40 milliseconds, all the lesions showed dramatic improvement in 1 week.

Smoothbeam® was also used to treat another patient with psoriatic plaques on the body who was using the same dosage of systemic cyclosporine and vitamin B3 as the first patient, and who had previously received pulse dye laser treatment with mild to moderate results. The treatment of the second patient with Smoothbeam® provided a dramatic improvement and was more effective than the results of pulse dye laser treatment.

### Example 3

### Treating Guttate Type Psoriasis

Patients with guttate type psoriasis were also treated with the 1450 nm laser system to alleviate the skin lesions.

### Example 4

### Treating Psoriasis With the Laser System Without Other Treatments

The laser system was also used alone, without the concurrent treatment using other modalities (such as cyclosporine or vitamin B3) to treat psoriasis (e.g., in Example 2).

### Example 5

### Treating Itch in Psoriasis

The Smoothbeam® laser system was used to treat itching symptoms of psoriasis. The treatment alleviated early itching lesions and prevented them from turning into new psoriatic lesions. Therefore, the long-lasting effects of the Smoothbeam® treatment of psoriasis may be due to its reduction of itch.

### Example 6

### Treating Lichen Simplex Chronicus and Chronic Dermatitis

The Smoothbeam® was also used to treat a few lichen simplex chronicus and chronic dermatitis cases (e.g., chronic dermatitis with thickening epidermis) with some success at the similar fluence. The treatment reduced the itching symptoms associated with chronic dermatitis with thickening epidermis.

The term “lesion,” is used in this disclosure and claims as a non-specific term referring to abnormal epidermal tissue.

A number of embodiments have been described. Nevertheless, it will be understood that various modifications may be made without departing from the spirit and scope of the disclosure. Accordingly, other embodiments are within the scope of the following claims.

What is claimed is:

1. A method of treating skin disorders in a preselected region of human skin, scalp and nails having at least one lesion characteristic of the disorder disposed therein, the method comprising the steps of:
(a) cooling an area of the skin above the preselected skin region; and
(b) applying energy to the preselected region, in the absence of an exogenously provided energy absorbing material, in an amount sufficient to ameliorate the lesion while keeping the temperature of the area of the skin above the preselected region below about 60 degrees Celsius before, during, or before and during the application of the energy.

2. The method of claim 1, wherein in step (b) the energy is provided by laser light or incoherent light.

3. The method of claim 1, wherein in step (b) the energy is provided by laser light.

4. The method of claim 3, wherein the laser light comprises a wavelength in the range from about 1.4 microns to about 2.5 microns.

5. The method of claim 3, wherein the laser light comprises a wavelength in the range from about 1.4 microns to about 1.5 microns.

6. The method of claim 5, wherein the laser light comprises a fluence in the range from about 2 joules to about 500 joules per square centimeter.

7. The method of claim 6, wherein the duration of laser light is in the range of 1.0 millisecond to 300 milliseconds.

8. The method of claim 5 or 7, wherein the duration of laser light is parted into multiple subpulses.

9. The method of claim 8, wherein the amount of subpulses is 4-5 subpulses.

10. The method of claim 9, wherein each created multiple subpulse has the duration range from 1 to 60 milliseconds.

11. The method of claim 9, wherein the interval between each subpulse is 2-6 milliseconds.

12. The method of claim 1, wherein step (a) occurs prior to step (b).

13. The method of claim 1 or 12, wherein step (a) occurs contemporaneously with step (b).

14. The method of claim 1, wherein the skin disorder treated is psoriasis.

15. The method of claim 14, wherein the treatment is directed to alleviating psoriasis itch.

16. The method of claim 1, wherein the energy system is Smoothbeam®.

17. The method of claim 1 or 16, wherein the treatment is given at a rate selected from the group consisting of: one time, once a week, two times per week, three times per week, every week, bi-weekly, tri-weekly or once a month.

18. The method of claim 1 or 16, wherein the treatment is combined with other known psoriasis treatments.

19. The method of claim 17, wherein the treatment is combined with other known psoriasis treatments.

20. The method of claim 1, wherein the skin disorder treated is chronic dermatitis or lichen simplex chronicus.
